(19) United States
(12) Patent Application Publication
(10) Pub. No.: US 2025/0307623 A1
(43) Pub. Date: Oct. 2, 2025

(54) OPTICAL APPARATUS FOR NEURAL NETWORK COMPUTATION

(71) Applicant: Yuan LU, Nancy (FR)

(72) Inventor: Yuan LU, Nancy (FR)

(21) Appl. No.: 18/987,176

(22) Filed: Dec. 19, 2024

Related U.S. Application Data
(60) Provisional application No. 63/570,297, filed on Mar. 27, 2024.

(51) Int. Cl. G06N 3/067; G06N 3/0464

(52) U.S. Cl. CPC G06N 3/067; G06N 3/0464

(57) ABSTRACT

Disclosed is an optical apparatus for neural network computation. A light emitting device receives a first input and a second input, and emits a light beam with a circular polarization rate corresponding to the first input and with an intensity corresponding to the second input. A computing apparatus includes a plurality of light emitting devices, and may further include a light mixer and a light polarization detection system. A sum of products of the plurality of input data INPUTᵢ (from second input) and respective weights Wᵢ (from first input) assigned to the plurality of input data is thus calculated by the computing apparatus by means of optical operation. The computing apparatus can be configured to perform convolution calculation, and serves as a node in a hidden layer of the neural network. And a computing system for performing neural network computation is thus realized by means of optical computing.

### CROSS-REFERENCE TO RELATED APPLICATIONS

This application claims the benefit of U.S. Provisional Application No. 63/570,297, filed on Mar. 27, 2024, the disclosure of which is incorporated herein by reference in its entirety and for all purposes.

### TECHNICAL FIELD

The disclosure herein relates to an optical apparatus for neural network computation.

### BACKGROUND

Deep neural networks have found success in a wide variety of applications, ranging from computer vision to natural language processing to game playing. Convolutional neural networks (CNNs), capitalizing on the spatial invariance of various image properties, have been especially popular in computer vision problems such as image classification, image segmentation, and even image generation. As performance on a breadth of tasks has improved to a remarkable level, the number of parameters and connections in these networks has grown dramatically, and the power and memory requirements to train and use these networks have increased correspondingly. Computational efficiency of CNNs now continues to be an active research area, and it remains difficult for embedded systems such as mobile vision, autonomous vehicles and robots, and wireless smart sensors to deploy CNNs due to the stringent constraints on power and bandwidth. To increase efficiency, many strategies have been employed to compress CNNs while maintaining performance, including pruning, trained quantization, Huffman encoding, and altered architectural design. On the hardware side, there are now specialized processing units for machine learning, such as IBM's TrueNorth chip, Movidius's vision processing units (VPUs), and Google's tensor processing units (TPUs).

Chang et al. has proposed a complementary strategy that incorporate a layer of optical computing prior to either analog or digital electronic computing, improving performance while adding minimal electronic computational cost and processing time. Optical computing is tantalizing for its high bandwidth, high interconnectivity, and inherently parallel processing, all potentially at the speed of light. Certain operations can be performed in free space or on a photonic chip with little to no power consumption, e.g. a lens can take a Fourier transform “for free”. An optimizable and scalable set of optical configurations that preserves these advantages and serves as a framework for building optical CNNs would be of interest to computer vision, robotics, machine learning, and optics communities. However, since their system involves many optical components (4f system), a large volume (^{˜}100 cm) prevents the system to be employed in a compact and portable imaging system. In addition, they use physical phase mask as filter for convolution, which cannot be changed electrically.

### SUMMARY

According to some embodiments of the disclosure, a light emitting device is provided, comprising: a first input component configured to receive a first input; a second input component configured to receive a second input; and a light emitting structure configured to emit a light beam with a circular polarization rate (PC) corresponding to the first input and with an intensity corresponding to the second input.

According to some embodiments of the disclosure, a computing apparatus is provided, which is configured to derive an output data from a plurality of first data and a plurality of second data, wherein the computing apparatus comprises a plurality of light emitting devices of the disclosure. Each of the plurality of light emitting devices is assigned with one of the plurality of first data and one of the plurality of second data. The light emitting structure of each of the plurality of light emitting devices emits a light beam with a circular polarization rate corresponding to one of the plurality of first data and an intensity corresponding to one of the plurality of second data. And, the output data is corresponding to the circular polarization rate of the mixed light beam obtained by mixing the light beams emitted by the plurality of light emitting devices.

According to some embodiments of the disclosure, a computing system for performing neural network computation is provided. The computing system comprises at least one computing apparatus of the disclosure. The computing apparatus is configured to perform an operation in which a sum of products of a plurality of input data and respective weights assigned to the plurality of input data is calculated. The first inputs of the respective light emitting devices of the computing apparatus are corresponding to the respective weights. And, the second inputs of the respective light emitting devices of the computing apparatus are respectively corresponding to the plurality of input data.

### BRIEF DESCRIPTION OF FIGURES

By more detailed description of the exemplary embodiments of the present disclosure in combination with the accompanying drawings, the above and other purposes, features and advantages of the present disclosure will become more apparent. In the exemplary embodiments of the present disclosure, the same reference numeral generally represents the same component.

FIG. 1 is a schematic view of the light emitting device according to an embodiment of the disclosure.

FIG. 2 is a cross-sectional view of an example of a multi-layer light emitting structure according to an embodiment of the disclosure.

FIG. 3 shows the selection rule of optical transition in direct band gap semiconductor quantum wells or quantum dots.

FIG. 4 is a schematic view of the light emitting device according to an embodiment of the disclosure.

FIG. 5 is a schematic view showing an example of relative positional relationships of the components of the light emitting device according to an embodiment of the disclosure.

FIG. 6 describes the magnetization switching in the injector Hall-bar structure by spin Hall effect (SHE).

FIG. 7A is a cross-sectional view of the light emitting device according to an embodiment of the disclosure.

FIG. 7B is a top view of the light emitting device according to an embodiment of the disclosure.

FIGS. 8A-8E schematically illustrate the magnetic domain flipping procedure with different number of current pulses applied into the spin injector channel.

FIG. 9 is a top view of an apparatus having a plurality of light emitting devices formed on one mesa according to an embodiment of the disclosure.

FIG. 10A shows an exemplified computing apparatus according to the disclosure.

FIG. 10B shows another exemplified computing apparatus according to the disclosure.

FIG. 11A is a schematic view of a computing apparatus according to an embodiment of the disclosure.

FIG. 11B is a schematic view of a computing apparatus according to another embodiment of the disclosure.

FIG. 12 is a brief schematic diagram of a computing system for performing neural network computation by means of optical computing according to an embodiment of the disclosure.

### DETAILED DESCRIPTION

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

In the disclosure, a new idea of optical neural networks (ONNs) is provided by using a computing apparatus having an array of light emitting devices (for example, light emitting diodes, i.e. LEDs). The circular polarizations of the light beams emitted from the light emitting devices are controlled by a first input, and the intensities of the light beams are controlled by a second input.

The final output of the computing apparatus is the circular polarization rate of the mixed light from the array of light emitting devices, which is the weighted sum of the circular polarizations of the light beams respectively emitted by each of the array of light emitting devices.

In embodiments, the light emitting devices can be formed by spin light emitting diodes (spin-LEDs). For each spin-LED, a spin injector injects spin-polarized carriers into the light emitting structure of the spin-LED. Stimulations (first input), such as electrical pulse stimulations, are used to switch the magnetization of the spin injector, which is then converted to the circular polarization of light emitted from the spin-LED.

This ONN can be used for convolution function in neural network. Each array of LEDs may have a synaptic function and the weights are stored in the magnetization of injectors. This structure of the disclosure can simultaneously benefit the non-volatile magnetic storage and fast computing speed of light.

In addition, the volume of LED is in μm size, the total structure can be embedded into compact mobile system.

Hereinafter, the light emitting device according to the disclosure will be described first.

FIG. 1 is a schematic view of the light emitting device according to an embodiment of the disclosure.

As shown in FIG. 1, the light emitting device 1 according to the disclosure includes a light emitting structure 10, a first input component 11 and a second input component 12.

In some embodiments, the light emitting structure 10 is a III-V (for example, GaAs, GaN), two-dimensional (2D) or perovskite semiconductor based structure for emitting light beam. In some further embodiments, the light emitting structure 10 can be a light emitting diode structure with gain medium of quantum wells or quantum dots.

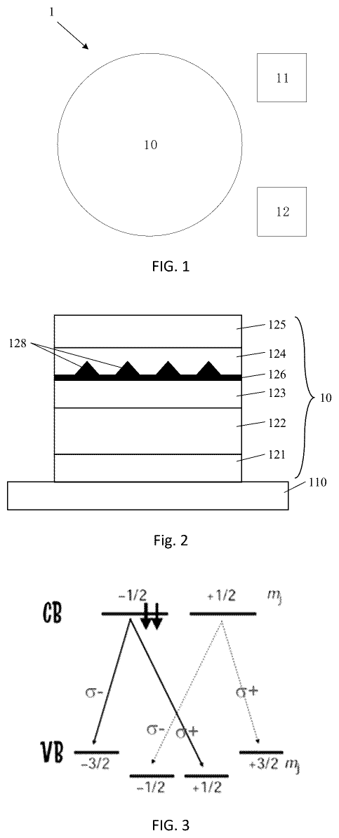

In some embodiments, the light emitting structure 10 may be in a form of a multi-layer structure, for example, the multi-layer light emitting structure 10 shown in FIG. 2.

FIG. 2 is a cross-sectional view of an example of a multi-layer light emitting structure according to an embodiment of the disclosure.

In some embodiments, the multi-layer light emitting structure 10 is a GaAs based structure. The multi-layer light emitting structure 10 can be a light emitting diode structure with a gain medium layer 128 formed above a wetting layer 126. Quantum wells or quantum dots are formed in the gain medium layer 128. P-doped layers (121, 122) are formed below the gain medium layer 128. And n-doped layer (125) is formed above the gain medium layer 128.

As shown in FIG. 2, multi-layer light emitting structure 10 is formed on a semiconductor substrate 110. The semiconductor substrate 110 might be a p-doped GaAs substrate with a (001) crystal plane, i.e., a p-GaAs (001) substrate.

In the example shown in FIG. 2, the multi-layer light emitting structure 10 includes, from bottom to top, a p-doped GaAs (p-GaAs) layer 121 (for example, 300 nm), a p-doped Al_{0.3}Ga_{0.7}As layer 122 (for example, 400 nm), a Be δ-doping GaAs layer 123 (for example, 30 nm), a wetting layer 126 of InGaAs with quantum wells or quantum dots 128 formed therein, an undoped GaAs layer 124 (for example, 50 nm) and a n-doped GaAs layer 125 (for example, 50 nm).

Back to FIG. 1, the first input component 11 is configured to receive a first input to control the circular polarization rate of the light beam emitted by the light emitting structure 10. The second input component 12 is configured to receive a second input to activate the light emitting structure 10 to emit a light beam and control the intensity of the light beam emitted by the light emitting structure 10.

Accordingly, the light emitting structure 10 is configured to emit a light beam with a circular polarization rate corresponding to the first input and with an intensity corresponding to the second input.

In other word, the first input can be used to control the circular polarization rate of the light beam emitted, and the second input can be used to control the intensity of the light beam emitted.

The light beam emitted from the light emitting structure 10 will contain two kinds of controlled information, i.e., the circular polarization rate and the intensity. When the circular polarization rate and the intensity of the light beam are respectively controlled by a first data and a second data (in other words, the circular polarization rate and the intensity of the light beam are respectively controlled to present the first data and the second data), the light emitting structure 10 can be used to perform a computation by involving the first data and the second data.

Hereinafter, it will be described in more details how to control the circular polarization rat and the intensity of the light beam emitted by the light emitting structure 10 according to the first input and the second input in embodiments. However, it shall be understood that the ways to control the circular polarization rate and the intensity of the light beam are not limited to the contents disclosed here. One skilled in the art shall know that there might be other manners to control the circular polarization rate and the intensity of the light beam emitted by the light emitting structure.

In embodiments of the disclosure, the first input is one or more current pulses. The circular polarization rate of the light beam emitted from the light emitting structure 10 is a function of the number and/or the magnitude and/or width (or duration) and/or direction of the one or more current pulses.

The functional relationship between the circular polarization rate with respect to the number and/or the magnitude and/or width (or duration) and/or direction of the one or more current pulses can be configured in advance through device structure design and experimental measurements.

In an embodiment, the magnitude, the width (or duration) and the direction of the one or more current pulses are configured to be identical, and the circular polarization rate of the light beam emitted from the light emitting structure 10 is a function of the number of the one or more current pulses. The magnitude of the current pulses will be close to the critical current for magnetization switching.

In embodiments of the disclosure, the second input is a voltage. The light emitting structure 10 emits light beam in response to the applied voltage (the second input), and the intensity of the light beam emitted from the light emitting structure 10 is a function of the magnitude of the voltage.

The functional relationship of the intensity of the light beam emitted from the light emitting structure 10 with respect to the magnitude of the voltage can be known in advance through experimental measurements.

In an embodiment, the intensity of the light beam emitted from the light emitting structure 10 is related to the magnitude of the voltage. The relationship between the intensity and the magnitude of the voltage can be configured in advance through device structure design and experimental measurements.

There are several ways to control circular polarization rate of the emitted light beam via a first input. Hereinafter, a way of controlling circular polarization rate of the emitted light beam by controlling spin polarization rate of the carriers injected into the light emitting structure 10 via a first input (for example, one or more current pulses) will be described.

Semiconductor spintronics technology will be very helpful to achieve the objective of emitting light with desired circular polarization. By depositing a ferromagnetic layer as a spin injection layer on the top of the quantum wells or quantum dots structure (for example, a PN junction with a structure similar to that of a light emitting diode (LED)), spin-polarized electrons can be injected into such a light emitting diode. The spin-polarized electrons will undergo quantum transition to recombine with holes according to the law of conservation of angular momentum, and thus circularly polarized photons will be emitted. Each of the semiconductor quantum wells or semiconductor quantum dots is capable of emitting photon with circular polarization direction determined by the spin direction of the injected spin-polarized carrier.

FIG. 3 shows the selection rule of optical transition in direct band gap semiconductor quantum wells or quantum dots.

As shown in FIG. 3, when an electron with spin of −½ is injected into the conduction band of a semiconductor quantum wells or quantum dots through a ferromagnetic spin injection layer or spin injector (such as CoFeB/MgO layer), according to the conservation law of angular momentum quantum number mⱼ (the change of angular momentum quantum number before and after the transition Δmⱼ=±1), the electron is allowed to transition to the valence band only in two ways.

One way is to transition with a heavy hole valence band (mⱼ=−3/2), that is, to transition from mⱼ=−½ to mⱼ=−3/2 (Δmⱼ=−1), emitting a left circularly polarized photon, which can be referred to as “σ−”.

The other way is to transition with a light hole valence band (mⱼ=+½), that is, to transition from mⱼ=−½ to mⱼ=+½ (Δmⱼ=+1), emitting a right circularly polarized photon, which can be referred to as “σ+”.

However, in the quantum well structure or the quantum dot structure, the light and heavy hole valence bands are non-degenerated, and the heavy hole transition matrix element (transition probability) is much higher than the light hole transition matrix element (transition probability). Therefore, while an electron with spin of −½ is injected, a left circularly polarized photon (σ−) will be obtained with almost 100% probability.

Conversely, while an electron with spin of +½ is injected, a right circularly polarized photon (σ+) will be obtained with almost 100% probability.

Therefore, the direction of circular polarization of the photon emitted from a quantum well or a quantum dot completely depends on the spin direction of the injected electron.

Since the light beam emitted from the light emitting structure 10 is made up of right circularly polarized photon (σ+) and left circularly polarized photon (σ−) generated in response to injected electrons with spin of +½ and −½ respectively, the circular polarization rate of the light beams emitted from the light emitting structure 10 closely corresponds to the spin polarization rate of the carriers injected into the light emitting structure 10.

It should be emphasized here that the optical selection rule requires the spin direction to be parallel to the photon emission direction. To obtain a circularly polarized photon without magnetic field, the magnetization direction of the ferromagnetic injection layer shall be perpendicular to the sample surface for surface emission geometry.

Based on the above principle, the light emitting structure 10 is capable of emitting light beam with controllable circular polarization by injecting carriers with controlled spin polarization rate into the light emitting structure 10.

The first input (one or more current pulses) can be used to control the spin polarization rate of the injected carriers.

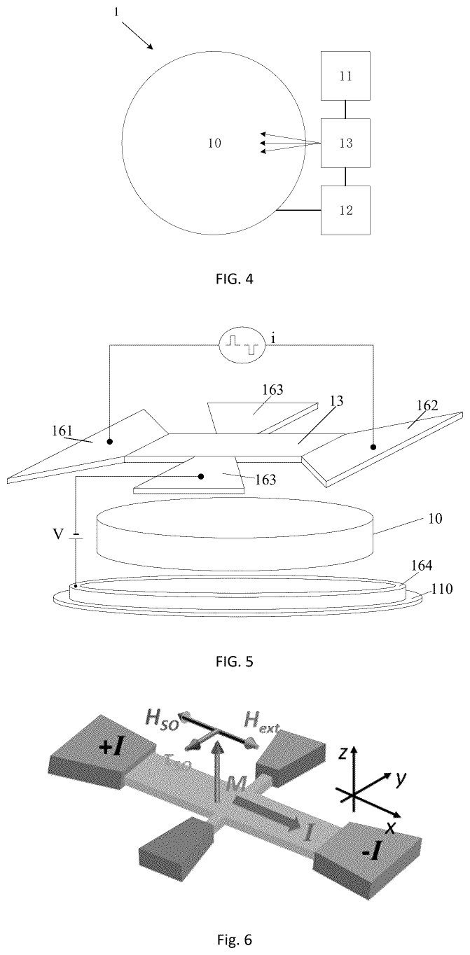

FIG. 4 is a schematic view of the light emitting device according to an embodiment of the disclosure.

As shown in FIG. 4, the light emitting device 1 may further include a spin injector 13. The spin injector 13 is configured to inject spin-polarized carriers into the light emitting structure 10.

Here, the carriers can be either electrons or holes. Generally, the electrons are used as the carriers because the spin lifetime of electrons is much longer than that of holes.

The spin injector 13 is connected with the first input component 11 to receive the first input. The spin-polarized carriers injected from the spin injector 13 have a spin polarization rate corresponding to the first input, for example, one or more current pulses.

The spin injector 13 is also connected with the second input component 12 to receive the second input. The spin injector 13 may inject spin-polarized carriers into the light emitting structure 10 in response to the second input, for example, a voltage applied between the spin injector 13 and the substrate on which the light emitting structure 10 is formed.

In response to the injected spin-polarized carriers, the light emitting structure 10 emits a light beam with a circular polarization rate corresponding to the spin polarization rate of the injected spin-polarized carriers, and with an intensity corresponding to the second input.

FIG. 5 is a schematic view showing an example of relative positional relationships of the components of the light emitting device according to an embodiment of the disclosure.

As shown in FIG. 5, a light emitting structure 10 is formed above a semiconductor substrate 110, and a spin injector 13 is formed above the light emitting structure 10.

In some embodiments, there might be some other layers sandwiched between the semiconductor substrate 110 and the light emitting structure 10. Or, in other embodiments, the light emitting structure 10 might be formed directly on the semiconductor substrate 110.

In some embodiments, there might be some other layers sandwiched between the light emitting structure 10 and the spin injector 13. Or, in other embodiments, the spin injector 13 might be formed directly on the light emitting structure 10.

In the example of FIG. 5, the spin injector 13 shown in FIG. 4 is depicted as a spin injector 13 in a form of a bar-shaped channel.

Further, the first input component 11 includes a first electrode 161 and a second electrode 162. The first electrode 161 and the second electrode 162 are respectively connected to two output terminals of a current pulse supplier to receive current pulses.

And, the first electrode 161 and the second electrode 162 may be formed above the light emitting structure 10, and are respectively connected to two opposite ends of the bar-shaped channel (spin injector 13) to apply one or more current pulses into the bar-shaped channel (spin injector 13) to electrically control the out-of-plane magnetization of the spin injector 13. In this example, as shown in FIG. 5, the first electrode 161 and the second electrode 162 are respectively connected to the two opposite ends in the lengthwise direction of the bar-shaped channel (spin injector 13), so as to introduce the current pulses into the bar-shaped channel to flow through the lengthwise direction.

In this example, the second input component 12 includes a third electrode 163 and a fourth electrode 164. The third electrode 163 and the fourth electrode 164 are respectively connected to two output terminals of a voltage signal supplier to receive voltage signals.

The third electrode 163 may be formed above the light emitting structure 10, and is connected to the bar-shaped channel (spin injector 13), and the fourth electrode 164 may be formed on the semiconductor substrate 110, so as to apply the voltage signal between the spin injector 13 and the semiconductor substrate 110.

In the example of FIG. 5, two of the third electrodes 163 are shown on two opposite sides in widthwise direction of the bar-shaped channel (spin injector 13). It shall be understood that the third electrode 163 can be formed in many other forms, as long as it is electrically connected to the spin injector 13.

In some embodiments, one or both of the first electrode 161 and the second electrode 162 can serve as the third electrode 163 to apply voltage signal to the spin injector 13. In other words, the first electrode 161 and/or the second electrode 162 can be further connected to one output terminal of the voltage signal supplier to receive the voltage signals, in addition to the current pulses.

There are several ways to control the spin polarization rate of the injected carriers via a first input. Hereinafter, a way of controlling the spin polarization rate of the injected carriers by controlling the magnetization state of the spin injector 13 via a first input (for example, one or more current pulses) will be described.

The spin polarization rate of the spin-polarized carriers injected from the spin injector 13 to the light emitting structure 10 can be determined by the magnetization state of the spin injector 13.

In some embodiments, the spin injector 13 is a metallic spin injector, and may have a Hall-bar structure. the magnetization state of the spin injector can be switched by spin Hall effect (SHE).

FIG. 6 describes the magnetization switching in the spin injector with a Hall-bar structure by SHE. A current/is injected in the designed ferromagnet (FM)/heavy metal (HM) spin-injector channel to generate current-induced spin-orbit torque (SOT) τ_{SO} and associated spin-orbit field H_{SO}. With a small in-plane external constant magnetic field Hₑₓₜ, the perpendicular magnetization of injector can be deterministically switched when injecting alternative direction of current in the channel. The magnetization switch can be realized with very short pulse current (6 ps), which allows for high-speed operation. The latest developments in the field of spintronics show the possibility to avoid the application of Hₑₓₜ by using different strategies, such as using spin textured ferromagnetic layer, an in-plane exchange bias or growth on substrates with specific crystalline orientation.

To electrically control the circular polarization rate of the emitted light beam, current pulses will be sent into the spin injector 13 (through the first electrode 161 and the second electrode 162) to switch the magnetization state of the spin injector 13. Then, the spin injector 13 will be negatively biased (through the third electrode 163 and the fourth electrode 164) to enable a light emission with an intensity corresponding to the bias voltage.

According to the optical selection rule, the circular polarization (right circular polarization σ+ and left circular polarization σ−) of the emitted light will be determined by the spin polarization of carriers injected from the spin injector 13. And the circular polarization rate of the light beam emitted from the light emitting structure 10 will be determined by the spin polarization rate of the carriers injected from the spin injector 13.

Therefore, by switching the magnetization state of the spin injector 13, the spin polarization rate of the injected carriers can be changed, and the circular polarization rate of the emitted light beam will be controlled accordingly.

In embodiments, the magnetization state of the spin injector 13 refers to the average out-of-plane magnetization amplitude of the magnetic domains.

Magnetization directions of magnetic domains in the spin injector 13 are flipped by applying the one or more current pulses into the bar-shaped channel of the spin injector 13. Due to incomplete flipping, some magnetic domains have up-direction (↑) magnetization, and some magnetic domains have down-direction (↓) magnetization. Hereinafter, the area of the magnetic domains having up-direction (↑) magnetization is referred to as “first area”, or “A^{↑}”, and the area of the magnetic domains having down-direction magnetization is referred to as “second area”, or “A^{↓}”.

The first area A^{↑} and the second area A^{↓} vary with the application of the one or more current pulses (the first input). In other words, the first area A^{↑} and the second area A^{↓} are functions of the number and/or the magnitude and/or width (or duration) and/or direction of the one or more current pulses.

In the embodiment where the magnitude, the width (or duration) and the direction of the one or more current pulses are identical, the first area A^{↑} and the second area A^{↓} are functions of the number of the one or more current pulses. As the number of the current pulses increases, more and more magnetic domains are flipped, the first area A^{↑} and the second area A^{↓} vary accordingly.

The normalized average out-of-plane magnetization amplitude of the magnetic domains is a ratio of a difference between the first area A^{↑} and the second area A^{↓} and a sum of the first area A^{↑} and the second area A^{↓}, i.e.,

$$\frac{{A}^{↑} - {A}^{↓}}{{A}^{↑} + {A}^{↓}} .$$

The magnetization states of the magnetic domains are non-volatile and are capable of being retained in the spin injector 13 after the one or more current pulses are applied into the spin injector 13.

In some embodiments, an initializing current pulse may be applied into the bar-shaped channel of the spin injector 13 before applying the one or more current pulses, to perform an initialization operation. By the initialization operation, the magnetic domains in the spin injector 13 are all flipped into an identical magnetization direction, for example, down-direction (↓). The current direction of the one or more current pulses (for example, from the first electrode 161 to the second electrode 162) is opposite to that of the initialization current pulse (for example, from the second electrode 162 to first electrode 161). And, by applying the one or more current pulses, a ratio of magnetic domains are flipped from down-direction (↓) magnetization to up-direction (↑) magnetization.

While a bias voltage (second input) is applied between the third electrode 163 and the fourth electrode 164, spin-polarized carriers are injected from the spin injector 13 to the semiconductor quantum wells or semiconductor quantum dots 128 in the light emitting structure 10.

The carriers injected from the magnetic domains having up-direction (↑) magnetization have a spin of +½, resulting right circularly polarized photons (σ+). And the carriers injected from the magnetic domains having down-direction (↓) magnetization have a spin of −½, resulting right circularly polarized photons (σ−).

Accordingly, the intensity I^{σ+} of light corresponding to the right hand circular polarization is proportional to the first area A^{↑} of the magnetic domains having up-direction (↑) magnetization, and the intensity I^{σ-} of light corresponding to the left hand circular polarization is proportional to the first area A^{↓} of the magnetic domains having down-direction (↓) magnetization.

Therefore, the circular polarization rate Pc of the light beam emitted from the light emitting structure 10 is determined by the normalized average out-of-plane magnetization of the magnetic domains, following the relationship:

$${P}_{c} = \frac{{I}^{σ +} - {I}^{σ -}}{{I}^{σ +} + {I}^{σ -}} = \frac{{A}^{↑} - {A}^{↓}}{{A}^{↑} + {A}^{↓}} .$$

It shall be understood that the total intensity I_{Total} of the light beam is a sum of intensities I^{σ+} and I^{σ-}:

$${I}_{Total} = {I}^{σ +} + {I}^{σ -} .$$

As the magnetization state changes in response to the application of the first input (current pulses), the spin polarization rate of the carriers injected from the spin injector 13 will change accordingly.

Accordingly, the circular polarization rate Pc of the light beam emitted from the light emitting structure 10, which is substantively equal to the spin polarization rate of the spin-polarized carriers injected from the spin injector 13 to the light emitting structure 10, is determined by a normalized average out-of-plane magnetization amplitude of the magnetic domains

$$\frac{{A}^{↑} - {A}^{↓}}{{A}^{↑} + {A}^{↓}} .$$

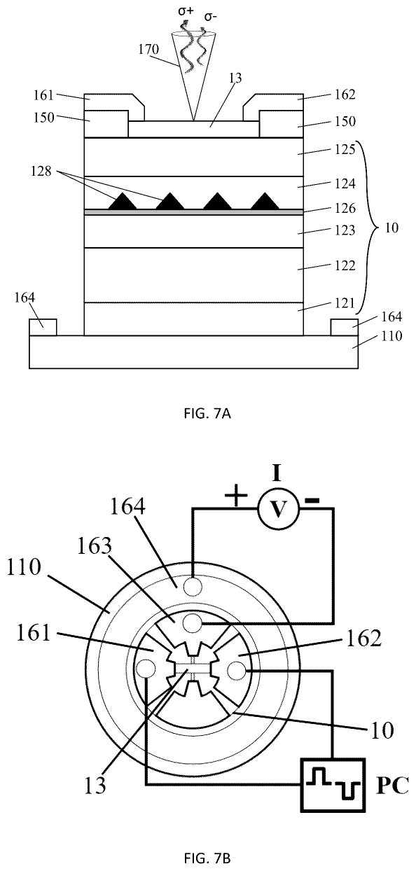

A more detailed structure of the light emitting device will be described below with reference to FIG. 7A and FIG. 7B.

FIG. 7A is a cross-sectional view of the light emitting device according to an embodiment of the disclosure. FIG. 7B is a top view of the light emitting device according to the embodiment of the disclosure.

As shown in FIG. 7A, a multi-layer light emitting structure 10 is formed on a semiconductor substrate 110. The semiconductor substrate 110 might be a p-doped GaAs substrate with a (001) crystal plane, i.e., a p-GaAs (001) substrate.

The multi-layer light emitting structure 10 includes, from bottom to top, a p-doped GaAs (p-GaAs) layer 121 (for example, 300 nm), a p-doped Al_{0.3}Ga_{0.7}As layer 122 (for example, 400 nm), a Be δ-doping layer 123 (for example, 30 nm), a wetting layer 126 of InGaAs, a gain medium layer 128 with quantum wells or quantum dots formed therein, a GaAs layer 124 (for example, 50 nm) and a n-doped GaAs layer 125 (for example, 50 nm).

In some embodiments, the multi-layer light emitting structure 10 is cylindrical, and when viewed from top down, it might be disc-shaped.

A spin injector 13 is formed on the top layer of the light emitting structure 10, i.e., the n-doped GaAs layer 125.

As described above, the spin injector 13 might be in form of a bar. The sizes (length and width) of the upper surface of the bar-shaped spin injector 13 are smaller than the radius of upper surface of the cylindrical light emitting structure 10.

A first electrode 161 and a second electrode 162, as well as a third electrode 163 (shown in FIG. 7B) are formed surrounding the spin injector 13, and connected with the spin injector 13.

The fourth electrode 164 is formed on the semiconductor substrate 110. The fourth electrode 164 might be a ring shape surrounding the cylindrical light emitting structure 10 with an interval.

As described above, the first electrode 161 and the second electrode 162 are respectively connected to two opposite ends of the bar-shaped channel of the spin injector 13 to apply one or more current pulses (first input) into bar-shaped channel of the spin injector 13. A current pulse generator (or a current pulse source) provides one or more current pulses corresponding to the circular polarization rate (PC) of the light beam desired to be emitted.

The third electrode 163 and the fourth electrode 164 are configured to apply a bias voltage between the spin injector 13 and the semiconductor substrate 110. A voltage source provides the bias voltage (V) corresponding to the intensity (I) of the light beam emitted.

In some embodiments, the first electrode 161 and/or the second electrode 162 may also serve as the third electrode 163 to receive the bias voltage with respect to the fourth electrode 164.

An insulating material layer 150 is formed between the electrodes 161, 162, 163 and the top layer of the light emitting structure 10 and surrounding the spin injector 13, insulating the electrodes 161, 162 and 163 from the top layer of the light emitting structure 10. The insulating material layer 150 might be formed by SiO₂.

In some embodiments, the first electrode 161, the second electrode 162, the third electrode 163 and the fourth electrode 164 are formed from Ti, or Au or combination of Ti and Au such as double-layer film (Ti/Au) and Ti—Au alloy.

By applying one or more current pulses (first input) into the spin injector 13 via the first electrode 161 and the second electrode 162, the magnetization state (average out-of-plane magnetization amplitude of the magnetic domains) of the spin injector 13 will change accordingly.

And then, by applying a bias voltage (second input) between the spin injector 13 and the semiconductor substrate 110 via the third electrode 163 and the fourth electrode 164, spin-polarized carriers will be injected from the spin injector 13 into the light emitting structure 10, especially in the quantum wells or quantum dots (gain medium layer 128).

In response to the spin-polarized carriers, the quantum wells or quantum dots 128 emits light with circular polarization (σ+ or σ−) corresponding to the spin polarization of the carriers.

And thus, the light emitting structure 10 will emit a light beam 170 with right circularly polarized portion (σ+) and left circularly polarized portion (σ−).

The intensity I^{σ+} of light corresponding to the right hand circular polarization is proportional to the first area A^{↑} of the magnetic domains having up-direction (↑) magnetization, and the intensity I^{σ-} of light corresponding to the left hand circular polarization is proportional to the first area A^{↑} of the magnetic domains having down-direction (↑) magnetization.

The total intensity I_{Total} of the light beam is a sum of intensities I^{σ+} and I^{σ-}:

$${I}_{Total} = {I}^{σ +} + {I}^{σ -} .$$

And, the total intensity I_{Total} of the light beam is determined by the amplitude of the bias voltage (second input) applied via the third electrode 163 and the fourth electrode 164.

The light beam 170 will have a circular polarization rate Pc determined by the normalized average out-of-plane magnetization of the magnetic domains of the spin injector 13, following the relationship:

$${P}_{c} = \frac{{I}^{σ +} - {I}^{σ -}}{{I}^{σ +} + {I}^{σ -}} = \frac{{A}^{↑} - {A}^{↓}}{{A}^{↑} + {A}^{↓}} .$$

And as described above, the normalized average out-of-plane magnetization of the magnetic domains of the spin injector 13 changes in response to the application of the current pulses (first input) applied via the first electrode 161 and the second electrode 162.

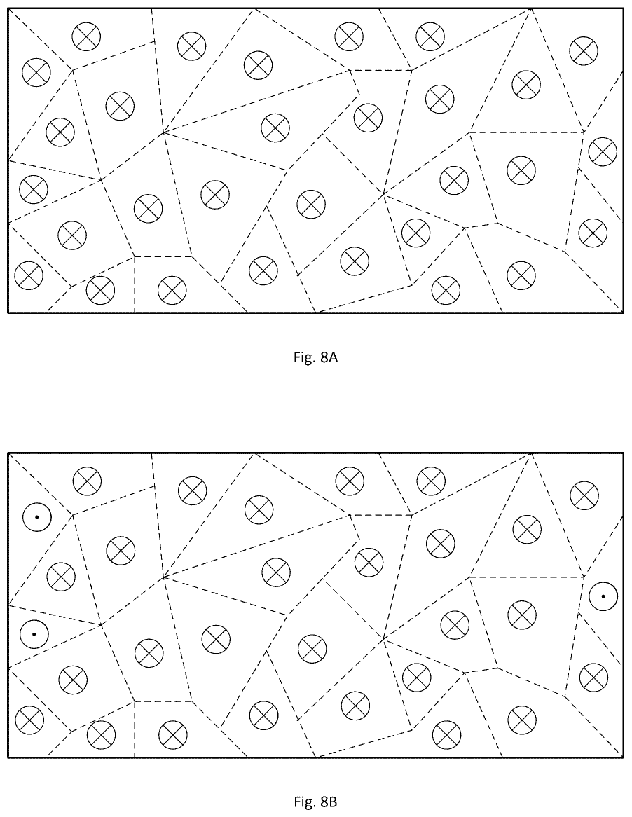

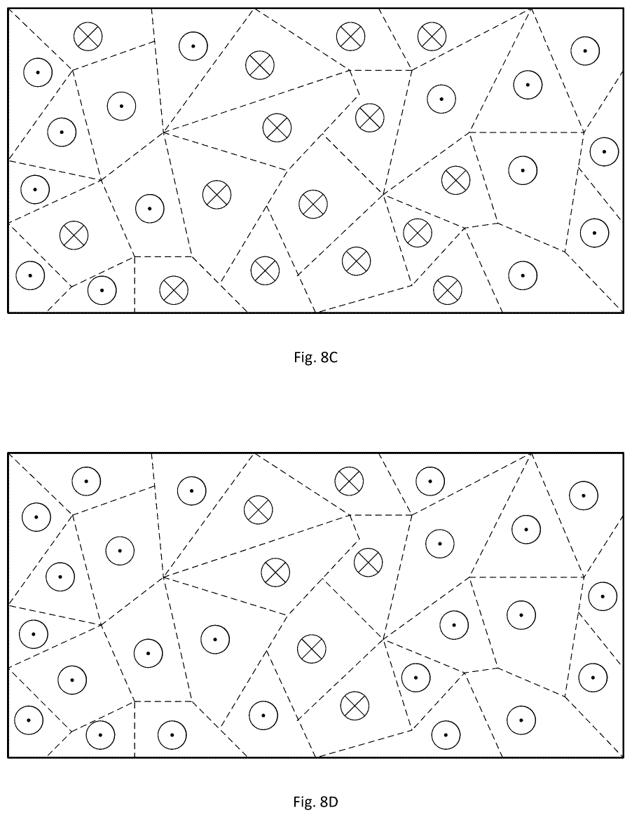

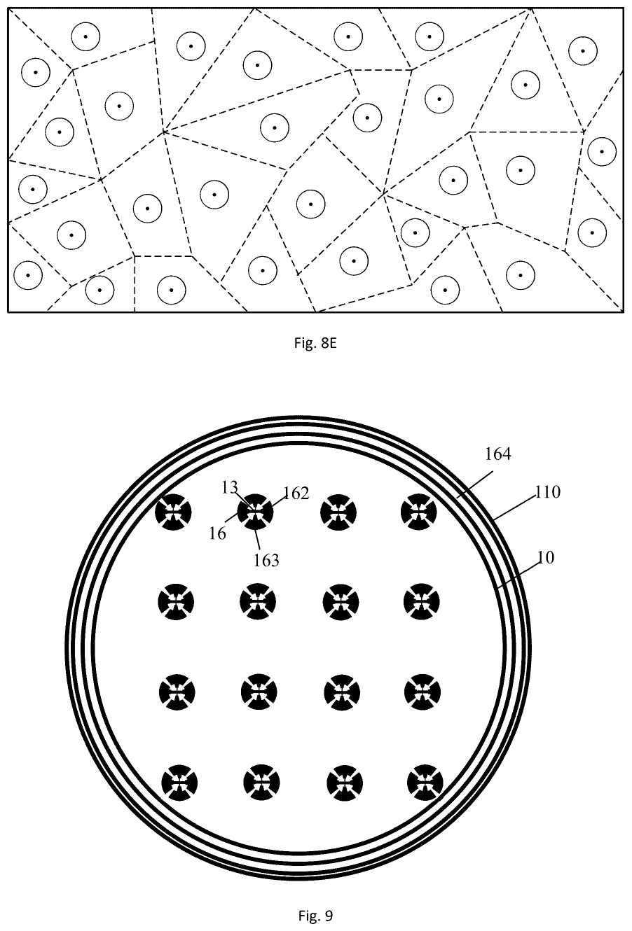

FIGS. 8A-8E schematically illustrate the magnetic domain flipping procedure with different number of current pulses applied into the spin injector channel. FIGS. 8A-8E are drawn as a top view of the spin injector 13. A circle with an X indicates that the magnetic domain has a down-direction (↑) magnetization, and a circle with a dot indicates that the magnetic domain has an up-direction (↓) magnetization.

The flipping operation may be performed at room temperature, with an in-plane magnetic field of Hx=+15 mT as an example. As an example, the pulse duration tₚᵤₗₛₑ of each current pulse is 1 ms and the amplitude Iₚᵤₗₛₑ of each current pulse is 45 mA.

The magnetization directions of magnetic domains will be flipped gradually with the increase of the pulse number. With a certain number of pulses (e.g. number of 10), the magnetic domains can be completely flipped.

FIG. 8A illustrates the magnetization directions of the magnetic domains after an initialization procedure. All the magnetic domains have a down-direction (↑) magnetization.

FIG. 8B illustrates the magnetization directions of the magnetic domains after applying 1 current pulse with a pulse duration tₚᵤₗₛₑ of 1 ms and an amplitude Iₚᵤₗₛₑ of 45 mA. About 10% of the magnetic domains are flipped into an up-direction (↓) magnetization.

FIG. 8C illustrates the magnetization directions of the magnetic domains after applying 5 current pulses with a pulse duration tₚᵤₗₛₑ of 1 ms and an amplitude Iₚᵤₗₛₑ of 45 mA. About half of the magnetic domains are flipped into an up-direction (↓) magnetization.

FIG. 8D illustrates the magnetization directions of the magnetic domains after applying 8 current pulses with a pulse duration tₚᵤₗₛₑ of 1 ms and an amplitude Iₚᵤₗₛₑ of 45 mA. About 80% of the magnetic domains are flipped into an up-direction (↓) magnetization.

FIG. 8E illustrates the magnetization directions of the magnetic domains after applying 10 current pulses with a pulse duration tₚᵤₗₛₑ of 1 ms and an amplitude Iₚᵤₗₛₑ of 45 mA. All of the magnetic domains are flipped into an up-direction (↓) magnetization.

To summarize, the magnetization state (average out-of-plane magnetization) of the spin injector 13 is controlled by the first input (for example, one or more current pulses), the spin polarization of the carriers injected into the light emitting structure 10 is determined by the magnetization state (average out-of-plane magnetization) of the spin injector 13, and the circular polarization rate of the light beam emitted from the light emitting structure 10 is consistent with the injected spin polarization of the carriers. Accordingly, the circular polarization rate of the light beam emitted from the light emitting structure 10 is controlled by the first input (for example, one or more current pulses).

Therefore, the light emitting structure is capable of emitting a light beam with circular polarization rate corresponding to the first input and with an intensity corresponding to the second input.

The light beam will carry two kinds of controllable information, the circular polarization rate and the intensity.

By assigning a plurality of discrete values of the circular polarization rate to a plurality of values of a first data, the first data (for example a weight W) with controllable values can be carried by the light beam.

And since the circular polarization rate of the light beam emitted from the light emitting structure 10 is corresponding to the first input, and the first input may have a set of parameters such as the number, the magnitude, the width (or duration) and the direction of the one or more current pulses (when the first input is one or more current pulses), the circular polarization rate is corresponding to a first parameter set of the first input, the first parameter set includes one or more first parameters.

When the first parameters are respectively assigned with discrete values (first parameter values), a plurality of discrete parameter value sets of the one or more first parameters will result in (and are corresponding to) a plurality of discrete values of the circular polarization rate with measurable differences.

In the embodiment where the magnitude, the width (or duration) and the direction of the one or more current pulses are identical, there will be only one controllable parameter, i.e. the number of the one or more current pulses. And the number of the current pulses applied into the spin injector 13 is corresponding to the plurality of discrete values of the circular polarization rate with measurable differences.

Accordingly, the plurality of values of the first data are corresponding to a plurality of discrete parameter value sets of the one or more first parameters (for example, the number of current pulses), and can be incorporated in the light beam by applying a first input with the discrete parameter value sets.

As a simple example, the first parameter set may have only one element, i.e., the number of the current pulses applied into the spin injector 13.

The first data, for example the weight, may be input to the system including the light emitting device 1 by controlling the parameter values in the first parameter set, i.e. the number of the current pulses.

Similarly, by assigning the plurality of discrete values of the intensity of the light beam to a plurality of values of a second data, the second data with controllable values can be carried by the light beam.

The intensity of the light beam emitted by the light emitting structure 10 is corresponding to a second parameter set of the second input, the second parameter set includes one or more second parameters.

A plurality of discrete parameter value sets of the one or more second parameters are corresponding to a plurality of discrete values of the intensity of the light beam with measurable differences.

In an embodiment, the second parameter set has only one element, i.e., a voltage value of the bias voltage applied between the spin injector 13 (connected to the third electrode 163) and the substrate 110 (connected to the fourth electrode 164).

The second data, for example the data to be processed, may be input to the system including the light emitting device 1 by controlling the parameter values in the second parameter set, i.e., the voltage of the bias voltage.

The light emitting device 1 of the present disclosure has been described in detail. As mentioned above, the light emitting device 1 may be referred to “spin-LED”.

The light emitting structure 10 may be in a form of multi-layer mesa. FIGS. 7A and 7B show only one light emitting device 1 formed on one multi-layer mesa with one spin injector 13.

In some embodiments, a plurality of light emitting devices 1 can be formed on one multi-layer mesa. The plurality of light emitting devices 1 share the multi-layer semiconductor mesa as the light emitting structure 10.

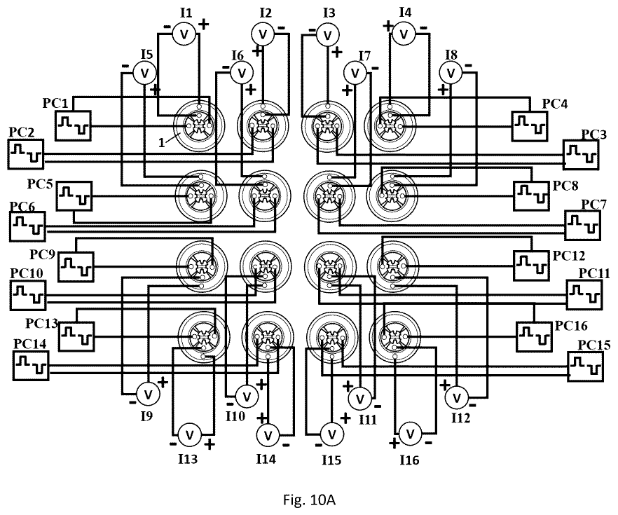

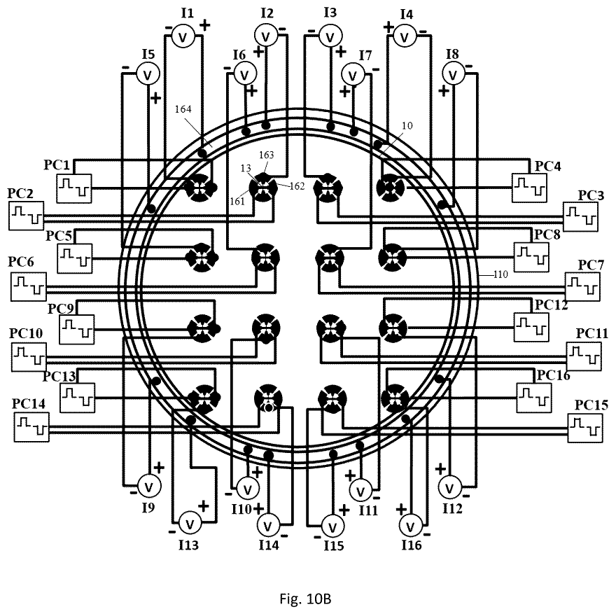

FIG. 9 is a top view of an apparatus having a plurality of light emitting devices formed on one mesa according to an embodiment of the disclosure.

As shown in FIG. 9, a light emitting structure 10 in a form of a mesa is formed on the semiconductor substrate 110, and can be shared by a plurality of (for example, an array of 4×4=16) light emitting devices. The light emitting structure 10 may have the same layers as those shown in FIG. 7A.

A plurality of the spin injectors 13, the first electrodes 161, the second electrodes 162 and the third electrodes 163 of the plurality of the light emitting devices are formed above the multi-layer semiconductor mesa. The plurality of light emitting devices 1 share the fourth electrode 164 connected to the semiconductor substrate 110.

By composing a plurality of (an array of) light emitting devices described above (for example, a plurality of light emitting devices as shown in FIG. 7B, or a plurality of light emitting devices formed in one apparatus as shown in FIG. 9), a computing apparatus can be provided to o derive an output data from a plurality of first data (for example, weight) and a plurality of second data.

In some embodiments, each spin-LED 1 may act as a synapse neuron.

Each spin-LED 1 has two inputs playing together on the output. For the two inputs, one (the first input) is the number of current pulse stimulation, which can change the status of magnetization in the spin injector 13 and determine the circular polarization rate of the emitted light beam. The synaptic property (non-linear response) can also be adjusted by the amplitude or width (or duration) of the current pulse. This parameter can be used as a weight of input (W). Once it is well optimized during the training procedure, it can be recorded in the spin injector 13 even without any retained power. It is non-volatile. The second input is the bias voltage for light emission. This parameter will determine the intensity of light (I) of each spin-LED.

In other words, each of the plurality of light emitting devices 1 is assigned with one of the plurality of first data (corresponding to the weight W) and one of the plurality of second data (corresponding to the intensity I of the light beam emitted by the light emitting device 1).

The light emitting structure 10 of each of the plurality of light emitting devices 1 emits a light beam with an intensity corresponding to one of the plurality of second data and a circular polarization rate corresponding to one of the plurality of first data, which might be the weight assigned to the one of the plurality of second data.

The first data assigned to the ith light emitting device 1 is corresponding to the circular polarization Wᵢ of the light beam emitted by the ith light emitting device 1, the second data assigned to the ith light emitting device 1 is corresponding to the intensity I; emitted by the ith light emitting device 1, wherein i is the index of the light emitting devices 1. And the total number of light emitting devices 1 in the computing apparatus is N, where N is a positive integer.

The average circular polarization (P̅C̅) of the mixed light beam by mixing the light beams emitted by the plurality of light emitting devices 1 can be obtained as:

$$\overline{P C} = \frac{{\sum }_{i = 1}^{N} {I}_{i} \cdot {PC}_{i}}{{\sum }_{i = 1}^{N} {I}_{i}}$$

The average circular polarization P̅C̅ of the mixed light beam can be used to derive the output data. And, the output data is corresponding to the circular polarization rate of the mixed light beam.

The final output, the circular polarization (P̅C) of the mixed light beam, can be regarded as a weighted sum of circular polarization PCᵢ of each spin-LED 1.

The first data Wᵢ assigned to the ith spin-LED 1 is proportional to the circular polarization PCᵢ, the second data INPUTᵢ assigned to the ith spin-LED 1 is proportional to the intensity Iᵢ, and the output data OUTPUT is proportional to the average circular polarization (P̅C). Thus, similar to the above equation, the output data OUTPUT meets the equation below:

$$OUTPUT = \frac{{\sum }_{i = 1}^{N} {INPUT}_{i} \cdot {W}_{i}}{{\sum }_{i = 1}^{N} {INPUT}_{i}}$$

This function can be used in the CNN either for the convolution calculation as a N (for example, N=m×n, where m and n are positive integers) pixel filter in a hidden layer or for the recognition function for classification in the output layer.

One array of spin-LEDs can be regarded as one node in the hidden or output layer in the CNN.

FIG. 10A shows an exemplified computing apparatus according to the disclosure.

As shown in FIG. 10A, the exemplified computing apparatus includes 16 light emitting devices 1 (spin-LEDs) as described above as shown in FIG. 7B.

FIG. 10B shows another exemplified computing apparatus according to the disclosure.

As shown in FIG. 10B, the exemplified computing apparatus includes 16 light emitting devices (spin-LEDs) formed in one apparatus as shown in FIG. 9. The 16 light emitting devices share one light emitting structure 10 in a form of multi-layer mesa. And further, the 16 light emitting devices share the fourth electrode 164 connected with the semiconductor substrate 110. The voltage (second input) is applied between the respective third electrodes 163 and the common fourth electrode 164.

In both FIG. 10A and FIG. 10B, the 16 spin-LEDs can be arranged in a 4×4 array. It shall be understood that the number of light emitting devices is not limited to 16. In general, the computing apparatus according to the disclose may include a plurality of light emitting devices 1.

As shown in FIG. 10A and FIG. 10B, for each of the plurality of light emitting devices, a first input (for example, one or more current pulses) corresponding to the circular polarization rate (PC) of the light beam emitted is received from a first source, for example, a current pulses generator. And the controllable circular polarization rate (PC) of the light beam is used to carry (or represent) a first data.

The first input component (for example, the first electrode 161 and the second electrode 162) of the light emitting device is configured to receive the first input. As described above, the first input has a first parameter value set (for example, the number of the current pulses), resulting a circular polarization rate (PC) of the light beam corresponding to the first data assigned to the light emitting device. The first data may be the weight assigned to this light emitting device.

Similarly, for each of the plurality of light emitting devices, a second input (for example, a bias voltage) corresponding to the intensity (I) of the light beam emitted is received from a second source, for example, a voltage source. And the controllable intensity (I) of the light beam is used to carry (or represent) a second data.

The second input component (for example, the third electrode 163 and the fourth electrode 164) of the light emitting device is configured to receive the second input. As described above, the second input has a second parameter value set (for example, the voltage value), resulting an intensity (I) of the light beam corresponding to the second data assigned to the light emitting device. The second data may be the input data to be processed by the light emitting device.

For the 16 light emitting devices 1, first inputs respectively resulting in circular polarization rates PC1, PC2, . . . , PC16 are received from the first sources, and second inputs respectively resulting in intensities I1, I2, . . . , I16 from the second sources.

The 16 circular polarization rates PC1, PC2, . . . , PC16 are corresponding to 16 first data (for example, weights) respectively assigned to the 16 light emitting devices 1, and the 16 intensities I1, I2, . . . , I16 are corresponding to 16 second data respectively assigned to the 16 light emitting devices 1.

The mixed light beam obtained by mixing the light beams emitted by the plurality of light emitting devices will have a circular polarization rate (P̅C̅):

$$\overline{P C} = \frac{{\sum }_{i = 1}^{N} {I}_{i} \cdot {PC}_{i}}{{\sum }_{i = 1}^{N} {I}_{i}}$$

And the mixed light beam will have a total intensity (I_{Total}):

$${I}_{Total} = {\sum }_{i = 1}^{N} {I}_{i}$$

Here, N is the number of light emitting devices 1 included in the computing apparatus, Iᵢ is the intensity of the light beam emitted by the ith light emitting device 1, PCᵢ is the circular polarization rate of the light beam emitted by the ith light emitting device 1.

Since the circular polarization rate and the intensity of the light beam emitted are assigned to the first data and the second data, a mathematical calculations of N pairs of first data Wᵢ and second data INPUTᵢ can be performed by an optical operation of mixing N light beams emitted by N light emitting devices 1:

$$OUTPUT = \frac{{\sum }_{i = 1}^{N} {INPUT}_{i} \cdot {W}_{i}}{{\sum }_{i = 1}^{N} {INPUT}_{i}}$$

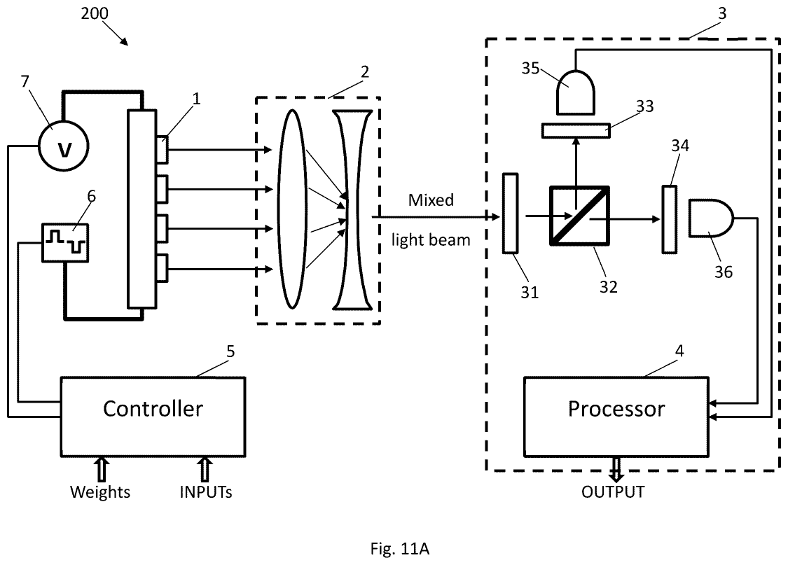

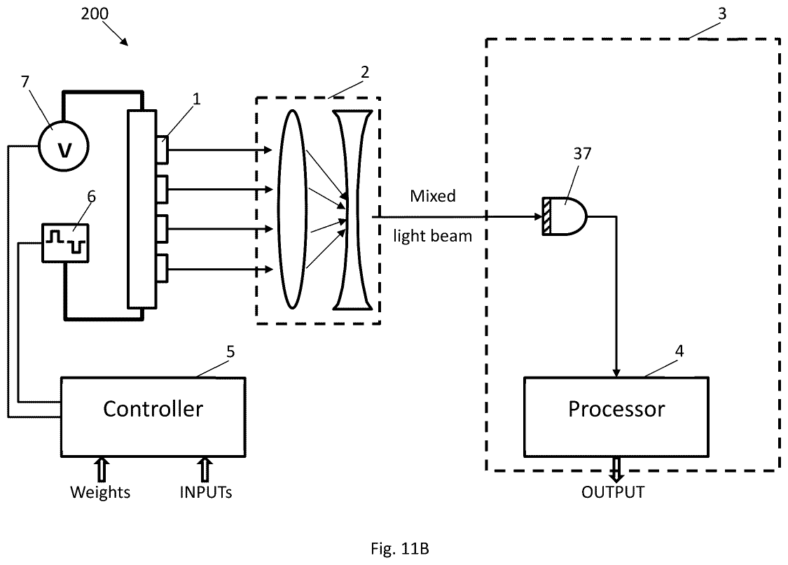

FIG. 11A is a schematic view of a computing apparatus according to an embodiment of the disclosure.

As shown in FIG. 11A, in the computing apparatus 200, a plurality of light emitting devices 1 emit light beams with a circular polarization rate corresponding to one of the plurality of first data and an intensity corresponding to one of the plurality of second data.

A controller 5 receives a plurality of (N) pairs of the first data (for example, weights Wᵢ) and the second data (for example, input data INPUTᵢ to be processed).

The controller 5 controls a first input source 6 (for example, one or more current pulse generators) to provide first inputs (for example, one or more current pulses) respectively to the first input components 161 and 162 of the N light emitting devices 1 according to the first data assigned to the respective light emitting devices.

The controller 5 controls a second input source 7 (for example, N bias voltage sources) to provide second inputs (for example, bias voltage) respectively to the second input components 163 and 164 of the N light emitting devices 1 according to the second data assigned to the respective light emitting devices.

Accordingly, the respective light emitting devices 1 will emit light beams with circular polarization rates corresponding to the respective first data and intensities corresponding to the respective second data.

The computing apparatus 200 further includes a light mixing component 2 and a light analyzer 3.

The light mixing component 2 may be a lens system (convex lens and concave lens) or an integrated Si photonic system, and is configured to mix the light beams emitted by the respective light emitting devices into a mixed light beam.

The light analyzer 3 is configured to obtain the circular polarization rate of the mixed light beam, and then, output an output data corresponding to the circular polarization rate of the mixed light beam.

As shown in FIG. 11A, the light analyzer 3 may include a ¼ waveplate 31, a beam splitter 32, two photodiodes 35, 36 and a processor 4.

In some embodiments, the processor 4 and the controller 5 are two separate devices. And in some other embodiments, the processor 4 and the controller 5 are implemented by the same device. The processor 4 can be also an analog circuit, which can directly convert the measured output circular polarization to a voltage, and the converted voltage can be provided to the second input component of a light emitting device of a computing apparatus as a second input, so as to perform further computation.

The ¼ waveplate 31 is configured to convert circular polarized light (left-hand polarized light and right-hand polarized light) of the mixed light beam into linear polarized light with two orthogonal linear polarization directions.

The beam splitter 32 is configured to split the mixed light beam into two light beams.

In some embodiments, the beam splitter 32 has polarization selectivity, such as Wollaston Prism (WP), and the two light beams have the two orthogonal linear polarization directions, respectively corresponding to the left-hand polarized component and the right-hand polarized component of the mixed light beam. In such embodiments, the polarizers 33 and 34 shown in FIG. 11A can be omitted. Or, the polarizers 33 and 34 may also be provided to enhance the filtering of undesired polarization components in the two split polarized light beams.

In some embodiments, the beam splitter can have no selectivity for polarization. The two light beams are identical in terms of polarization state. In other words, the proportions of the components of the two light beams in the aforementioned two orthogonal linear polarization directions are the same.

The polarizers 33 and 34 are respectively set in the aforementioned two orthogonal polarization directions to filter the light and result in the light with the two orthogonal linear polarization directions.

The two photodiodes 35 and 36 are respectively configured to detect light intensities of the two light beams with orthogonal linear polarizations. The two photodiodes 35 and 36 send electrical signals corresponding to the intensities of the two light beams to the processor 4.

The processor 4 is configured to read the electrical signals from the two photodiodes and obtain the circular polarization rate of the mixed light beam based on the intensities of the two light beams, and derive the output data corresponding to the circular polarization rate.

FIG. 11B is a schematic view of a computing apparatus according to another embodiment of the disclosure.

The embodiment illustrated in FIG. 11B varies from that in FIG. 11A with respect to the configuration of the light analyzer 3.

As shown in FIG. 11B, the light analyzer 3 may include a spin photodiode 37 and a processor 4.

The spin photodiode receives directly circularly polarized light and generates spin-related photocurrent corresponding to the circular polarization.

The processor 4 is configured to read the spin-related photocurrent from the spin photodiode 37, obtain the circular polarization rate of the mixed light beam, and derive the output data based on the circular polarization rate.

It can be seen that a plurality of (N, the number of the light emitting devices) pairs of first data (for example, weights Wᵢ) and the second data (for example, input data INPUTᵢ to be processed) are input into the computing apparatus 200, and an output data OUTPUT is derived through optical operation, or optical computing:

$$OUTPUT = \frac{{\sum }_{i = 1}^{N} {INPUT}_{i} \cdot {W}_{i}}{{\sum }_{i = 1}^{N} {INPUT}_{i}}$$

The computing apparatus according to the disclosure can be configured to perform convolution calculation.

In embodiments for convolution calculation, the computing apparatus serves as a convolution kernel calculator, the number of the light emitting devices 1 is equal to the number of the elements in the convolution kernel. The values of the elements in the convolution kernel are respectively assigned to the plurality of light emitting devices 1 as the first data. And the pixel values to be processed are respectively assigned to the plurality of light emitting devices as the second data.

On the other hand, the computing apparatus can be configured to perform computation of a node in a hidden layer of a neural network.

The node is connected with a plurality of nodes in the previous layer of the neural network. For example, the node can be connected with all of the nodes in the previous layer.

The number of the light emitting devices 1 in the computing apparatus is equal to the number of nodes in the previous layer connected with the node of the computing apparatus.

Each light emitting device 1 in the computing apparatus receives a first input corresponding to a weight Wᵢ (first data) to control the magnetization state of the spin injector 13, and receives a second input corresponding to the output data of a node of the previous layer (input data INPUTᵢ of the node of the computing apparatus, second data).

A sum of products of the plurality of input data INPUTᵢ and respective weights Wᵢ assigned to the plurality of input data is thus calculated by the computing apparatus.

Based on the computing apparatus of the disclosure, a computing system for performing neural network computation by means of optical computing can be realized.

As described above, the computing apparatus in the computing system can be configured to perform convolution calculation, or the computing apparatus is configured to perform computation of a node in a hidden layer of the neural network.

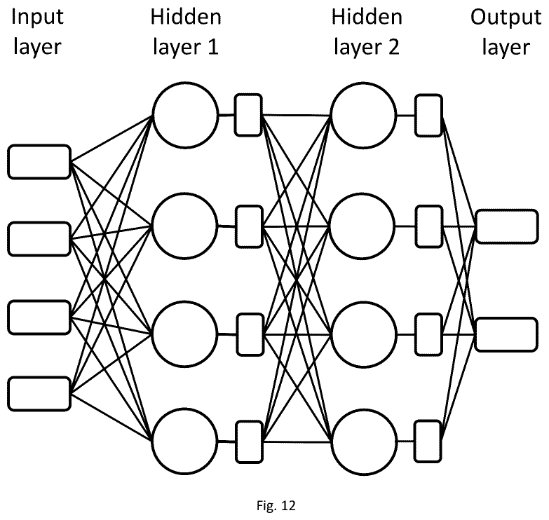

As an example, a computing system in which the computing apparatus of the disclosure is configured to perform computation of a node in a hidden layer of the neural network will be described below with reference to FIG. 12.

FIG. 12 is a brief schematic diagram of a computing system for performing neural network computation by means of optical computing according to an embodiment of the disclosure.

As show in FIG. 12, the neural network includes an input layer, hidden layer 1, hidden layer 2, and an output layer.

The computation of at least one node of at least one hidden layer is performed by means of the computing apparatus of the disclosure.

The computing apparatus is configured to perform an operation in which a sum of products of a plurality of input data and respective weights assigned to the plurality of input data is to be calculated.

The first inputs of the respective light emitting devices of the computing apparatus are corresponding to the respective weights. And, the second inputs of the respective light emitting devices of the computing apparatus are respectively corresponding to the plurality of input data. Here, the weights and the input data are respectively the first data and the second data as described above.

The input layer receives input data. And the input data are respectively converted to second inputs (for example, bias voltages) accordingly.

For any node performing computation by means of the computing apparatus of the disclosure, first inputs (for example, one or more current pulses) corresponding to the first data (for example, weights) are applied to the respective first input components of the plurality of light emitting devices of the computing apparatus, so as to respectively control the magnetization states of the spin injectors of the plurality of light emitting devices, and thus, “store” the respective first data (for example, weights) in the light emitting devices.

In the node, the weights are respectively assigned to the input data from the nodes in the previous layer of the neural network. And the weights can be determined by a training procedure performed previously.

The second inputs are applied to the computing apparatus for respective nodes in the hidden layer 1 based on the output of the nodes in the previous layer in the neural network.

The output data of the nodes in the previous layer are converted into second inputs (bias voltages) and applied to the respective second input components of the plurality of light emitting devices of the computing apparatus of the node, so the light emitting devices emit light beams with circular polarization rates corresponding to the first inputs and intensities corresponding to the second inputs.

The circular polarization rate of the mixed light beam is obtained as described above, and the output data of the node is derived accordingly, which is the sum of products of a plurality of input data and respective weights assigned to the plurality of input data, divided by the sum of a plurality of input data.

The output data of the node can be further converted to a second input (for example, a bias voltage), and then applied to a node in a following layer of the neural network for further optical computation.

In FIG. 12, in the hidden layers, the circles indicate the computation procedure deriving an output data from first inputs corresponding to first data (for example, weights) stored previously and second inputs corresponding to second data currently input from all nodes in the previous layer of the neural network. And the blocks indicate the conversion procedure converting the output data of the nodes to second inputs (for example, bias voltages) accordingly.

In the computing system, the computing apparatus can be configured to perform computation of a node of a fully connected layer of the neural network.

The number of the plurality of light emitting devices comprised in one computing apparatus equals to the number of nodes in a previous layer of the neural network.

On the other hand, if the computing apparatus is configured to perform convolution calculation, the number of the plurality of light emitting devices comprised in one computing apparatus equals to the number of elements in a convolution kernel for the convolution calculation.

In some embodiments, one computing apparatus of the disclosure is assigned to only one node. And, the number of the computing apparatus in the computing system equals to or larger than the number of the nodes in the fully connected layer(s). For every node in the fully connected layer(s), an exclusive computing apparatus of the disclosure is assigned to perform optical computation.

In some other embodiments, one computing apparatus of the disclosure may be assigned to more than one node. The number of the computing apparatus in the computing system is less than the number of the nodes in the fully connected layer(s). And thus, one computing apparatus is configured to perform computation of a plurality of nodes of one or more fully connected layers of the neural network.

It can be understood that the whole neural network as shown in FIG. 12 can be configured to realize, for example, a recognition function.

CNN and their applications appear to be spreading through all aspects of current lives, from self-driving cars to face recognition to medical diagnostics. The complex models require previously unfathomable power demands, making certain usages impractical or impossible, especially in embedded systems. Efforts to reduce complexity and increase speed while maintaining performance are being urgently required both in software and hardware.

Incorporation of optical computing would significantly accelerate the CNN computing and learning process while relieving the energy consumption from software and electronic hardware.

Embodiments of the disclosure, by using spin-LED (light emitting devices 1) to realize an ONN embedded CNN, will have following advantages.

Trained weight (first data) is stored in magnetic injector, which is kept without any power consumption. The weight parameters can be changed electrically with high speed pulse (in ns range).

The recognition and convolution in ONN (optical computation) are realized in the speed of light, which is much faster than using normal computer.

The information encoded in the light can be transmitted to another place with a long distance (through free space or coupled optical fiber). This can be used for non-local processing the CNN information by benefiting the non-local large serve with high processing speed.

The spin-LED arrays can be realized in an 8″ inch wafer to realize large number of neuron. With diameter of LED mesa about 2-3 μm, it is estimated to have 300 million LEDs to realize very complicated CNN structure. The volume of system can be also much reduced by using compact ¼ waveplate/polarizer/photodetector module, which can fulfill the needs of practical applications. It is also possible to use a spin-photodiode to detect the circular polarization rate, which can further reduce the volume of system.

Optical computing has potential for lower latency than electronic computation, which could be highly valuable in interactive systems such as autonomous vehicles where fast decision-making is crucial. Additionally, optical implementations have the potential to expand beyond traditional operations of CNNs, perhaps by harnessing wave optics and quantum optics in new ways. Our innovation thus offers new and valuable insights into the potential of optical CNNs in the future of high-efficiency autonomous computer vision systems.

Embodiments of the light emitting device, the computing apparatus and computing system of the disclosure is described in detail.

Those skilled in the art may understand that appropriate modifications can be made to various above-described circuit structures of the present disclosure as needed, all of which are within the scope of protection of the present disclosure.

Various embodiments of the present disclosure have been described above, and the foregoing descriptions are exemplary, not exhaustive, and not limiting of the disclosed embodiments. Numerous modifications and variations will be apparent to those of ordinary skill in the art without departing from the scope and spirit of the described embodiments. The terminology used herein was chosen to best explain the principles of the various embodiments, the practical application or improvement over the technology in the marketplace, or to enable others of ordinary skill in the art to understand the various embodiments disclosed herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A light emitting device comprising:
a first input component configured to receive a first input;
a second input component configured to receive a second input; and
a light emitting structure configured to emit a light beam with a circular polarization rate corresponding to the first input and with an intensity corresponding to the second input.

2. The light emitting device according to claim 1, wherein the first input is one or more current pulses, the circular polarization rate of the light beam emitted from the light emitting structure is a function of the number and/or the magnitude and/or width and/or direction of the one or more current pulses.

3. The light emitting device according to claim 2, further comprising:
a spin injector configured to inject spin-polarized carriers into the light emitting structure, wherein the spin-polarized carriers have a spin polarization rate corresponding to the first input,
in response to the injected spin-polarized carriers, the light emitting structure emits the light beam with the circular polarization rate corresponding to the spin polarization rate of the injected spin-polarized carriers.

4. The light emitting device according to claim 3, wherein the spin injector is in a form of a bar-shaped channel, the first input component comprises a first electrode and a second electrode respectively connected to two opposite ends of the bar-shaped channel to apply the one or more current pulses into the bar-shaped channel to electrically control the out-of-plane magnetization of the spin injector,
wherein the spin polarization rate of the spin-polarized carriers injected from the spin injector to the light emitting structure is determined by the magnetization state of the spin injector.

5. The light emitting device according to claim 4, wherein
magnetization directions of magnetic domains in the spin injector are flipped by applying the one or more current pulses into the bar-shaped channel of the spin injector, and due to incomplete flipping, a first area of magnetic domains has up-direction magnetization and a second area of magnetic domains has down-direction magnetization, and the first area and the second area vary with the application of the one or more current pulses,
the spin polarization rate of the spin-polarized carriers injected from the spin injector to the light emitting structure is determined by an average out-of-plane magnetization amplitude of the magnetic domains, and the average out-of-plane magnetization amplitude of the magnetic domains is a ratio of a difference between the first area and the second area and a sum of the first area and the second area.

6. The light emitting device according to claim 5, wherein the magnetic domains are non-volatile and are capable of being retained in the spin injector after the one or more current pulses are applied into the spin injector.

7. The light emitting device according to claim 4, wherein the second input is a voltage, the intensity of the light beam emitted from the light emitting structure is a function of the magnitude of the voltage.

8. The light emitting device according to claim 7, further comprising:
a semiconductor substrate, wherein the light emitting structure is formed above the semiconductor substrate, and the spin injector is formed above the light emitting structure,
the second input component comprises a third electrode and a fourth electrode respectively connected to the spin injector and the semiconductor substrate and configured to apply the voltage between the spin injector and the semiconductor substrate to inject carriers into the light emitting structure from the spin injector.

9. The light emitting device according to claim 1, wherein the circular polarization rate is corresponding to a first parameter set of the first input, the first parameter set includes one or more first parameters,
a plurality of discrete parameter value sets of the one or more first parameters are corresponding to a plurality of discrete values of the circular polarization rate with measurable differences, and
the plurality of discrete values of the circular polarization rate are assigned to a plurality of values of a first data.

10. The light emitting device according to claim 1, wherein the intensity is corresponding to a second parameter set of the second input, the second parameter set includes one or more second parameters,
a plurality of discrete parameter value sets of the one or more second parameters are corresponding to a plurality of discrete values of the intensity of the light beam with measurable differences, and
the plurality of discrete values of the intensity of the light beam are assigned to a plurality of values of a second data.

11. The light emitting device according to claim 1, wherein the light emitting structure comprises semiconductor quantum wells or semiconductor quantum dots, each of the semiconductor quantum wells or semiconductor quantum dots is capable of emitting photon with circular polarization direction determined by the spin direction of the injected spin-polarized carrier.

12. An apparatus comprising a plurality of light emitting devices according to claim 8, wherein
the apparatus comprises a multi-layer semiconductor mesa, the plurality of light emitting devices share the multi-layer semiconductor mesa as the light emitting structure,
the spin injectors, the first electrodes, the second electrodes and the third electrodes of the plurality of the light emitting devices are formed above the multi-layer semiconductor mesa, and
the plurality of light emitting devices share the fourth electrode connected to the semiconductor substrate of the multi-layer semiconductor mesa.

13. A computing apparatus configured to derive an output data from a plurality of first data and a plurality of second data, wherein the computing apparatus comprises a plurality of light emitting devices according to claim 1,
each of the plurality of light emitting devices is assigned with one of the plurality of first data and one of the plurality of second data,
the light emitting structure of each of the plurality of light emitting devices emits a light beam with a circular polarization rate corresponding to one of the plurality of first data and an intensity corresponding to one of the plurality of second data, and
the output data is corresponding to the circular polarization rate of the mixed light beam obtained by mixing the light beams emitted by the plurality of light emitting devices.

14. The computing apparatus according to claim 13, wherein for each of the plurality of light emitting devices,
the first input component of the light emitting device is configured to receive the first input with the first parameter value set, resulting a circular polarization rate of the light beam emitted corresponding to the first data assigned to the light emitting device;
the second input component of the light emitting device is configured to receive the second input with the second parameter value set, resulting an intensity of the light beam emitted corresponding to the second data assigned to the light emitting device.

15. The computing apparatus according to claim 14, further comprising:
a light mixing component configured to mix the light beams emitted by the respective light emitting devices into a mixed light beam; and
a light analyzer configured to obtain the circular polarization rate of the mixed light beam, and output the output data corresponding to the circular polarization rate of the mixed light beam.

16. The computing apparatus according to claim 15, wherein
the light analyzer comprising:
a ¼ waveplate configured to convert circular polarized photons of the mixed light beam into linear polarized photons with two orthogonal linear polarization directions;
a beam splitter configured to split the mixed light beam into two light beams, wherein two polarizers are respectively configured to filter the light with the two orthogonal linear polarization, and/or the beam splitter directly split the mixed light beam into two light beams with the two orthogonal linear polarization directions, respectively corresponding to the left polarized component and the left polarized component of the mixed light beam;
two photodiodes configured to detect light intensities of the two light beams with orthogonal linear polarizations; and
a processor configured to read the electrical signals from the two photodiodes and obtain the circular polarization rate of the mixed light beam based on the intensities of the two light beams, and derive the output data based on the circular polarization rate,
or, the light analyzer comprising:
a spin photodiode configured to receive directly circularly polarized light and generates spin-related photocurrent corresponding to the circular polarization,
a processor configured to read the spin-related photocurrent from the spin photodiode, obtain the circular polarization rate of the mixed light beam, and derive the output data based on the circular polarization rate.

17. The computing apparatus according to claim 13 further comprising:
a controller configured to provide the first inputs respectively to the first input components of the plurality of light emitting devices according to the first data assigned to the respective light emitting devices, and to provide the second inputs respectively to the second input components of the plurality of light emitting devices according to the second data assigned to the respective light emitting devices.

18. The computing apparatus according to claim 13, wherein
the computing apparatus is configured to perform convolution calculation, or
the computing apparatus is configured to perform computation of a node in a hidden layer of a neural network.

19. A computing system for performing neural network computation, wherein the computing system comprises at least one computing apparatus according to claim 13,
the computing apparatus is configured to perform an operation in which a sum of products of a plurality of input data and respective weights assigned to the plurality of input data is calculated,
the first inputs of the respective light emitting devices of the computing apparatus are corresponding to the respective weights, and the second inputs of the respective light emitting devices of the computing apparatus are respectively corresponding to the plurality of input data.

20. The computing system according to claim 19, wherein
the number of the plurality of light emitting devices comprised in one of the at least one computing apparatus equals to the number of elements in a convolution kernel for the convolution calculation, or
the number of the plurality of light emitting devices comprised in one of the at least one computing apparatus equals to the number of nodes in a previous layer of the neural network.
